(19) United States
(12) Patent Application Publication
(10) Pub. No.: US 2003/0073674 A1
(43) Pub. Date: Apr. 17, 2003

(54) Agents and methods for the prevention of initial onset of cancers, the treatment of cancers, and the recurrence of existing cancers

(75) Inventors: Thomas J. Slaga, Golden, CO (US); Addanki P. Kumar, Denver, CO (US); William Alworth, New Orleans, LA (US)

Correspondence Address: DAVID G. HENRY, 900 Washington Avenue, P.O. Box 1470, Waco, TX, 76701, US

(73) Assignee: Oncology Sciences Corporation, Austin, TX (US)

(21) Appl. No.: 10/245,775

(22) Filed: Sep. 17, 2002

Related U.S. Application Data
(63) Continuation of application No. PCT/US01/08718, filed on Mar. 19, 2001.
(63) Continuation-in-part of application No. 09/780,269, filed on Feb. 9, 2001, now Pat. No. 6,518,261.
(63) Continuation-in-part of application No. 09/777,151, filed on Feb. 5, 2001.

(51) Int. Cl. A61K 31/56

(52) U.S. Cl. 514/171

(57) ABSTRACT

The use of 2-methoxyestradiol, analogues of 2-methoxyestradiol, their method of synthesis and therapeutic use, and the use of combinations of the 2 methoxyestradiol and its analogues, with and without synergistic compounds (namely eugenol), all in the prevention of initial onset cancers, treatment of existing cancers, and the recurrence of previously existing cancers.

### CITATION TO PRIOR APPLICATION

This is a continuation of PCT/US 0,108,718 (published in English on Sep. 17, 2001—International Publication No. WO 01/70093 A2) designating the United States and with an International Filing Date of Mar. 19, 2001, and a Priority Date of Mar. 17, 2000 (based on U.S. application Ser. No. 09,527,283, now abandoned). This is also a continuation-in-part of co-pending U.S. application Ser. No. 09,780,269, filed Feb. 9, 2001, and of co-pending U.S. application Ser. No. 09,777,151, filed Feb. 5, 2001. Applicant claims priority, as applicable, pursuant to 35 U.S.C. §§ 119-120, and as provided under the Patent Cooperation Treaty.

### BACKGROUND OF THE INVENTION

A. Field of the Invention

The present invention concerns the novel use of known chemical compounds, the chemical synthesis and use of novel chemical compounds, and the use of novel combinations of both new and existing compounds, all in the treatment, prevention of initial onset and recurrence of a broad array of cancers.

B. Background of the Invention

1. The Problem: Primary Modalities of Cancer Cell Growth and Expansion (New, Existing and Recurring Cancers)

Cancer is the second leading cause of death in the United States, accounting for approximately one in four deaths. Recent estimates by the American Cancer Society suggest that in excess of 500,000 people die from cancer every year—that is approximately 1,500 deaths a day. Further, approximately 2.5 million new cases of cancer were expected to be diagnosed in the year 2000 alone. At an estimated annual cost of $107 billion dollars in health care costs and lost productivity due to death and illness, cancer inflicts a vast human and monetary toll on the United States.

The generic use of the term “cancer” only hints at the vast diversity of anatomical structures that this disease affects and the myriad of molecular bases that form the foundation of this disease. The collective use of the word cancer includes diseases affecting the brain, breast, cervix uteri, colon, corpus uteri, kidney, renal pelvis, larynx, lung, bone marrow, bronchus, skin, lymph system, nervous system, oral cavity, pharynx, ovary, pancreas, prostate, rectum, stomach, testis, thyroid, urinary bladder, and others. The individual molecular bases of these diverse afflictions can be varied and diverse. However, among this diverse field of afflictions, there exist two unified modalities of cell growth and/or proliferation that are common to almost all types of cancer: 1) unchecked cell growth and/or immortality, and 2) angiogenesis.

On of the problems that characterize a vast number of cancers is the unregulated growth or unchecked life span of aberrant cells in the various tissues of the body. Normal cells grow, divide, and die on a regular basis. The process by which cells normally die is called apoptosis. However, when normal cell growth and death become unchecked in the body, by any number of processes, such unchecked growth and/or immortality leads to destroy the regular functioning of the various tissues of the body. Such growth or immortality can ultimately lead to the occurrence of a host of solid tumors, leukemia's, lymphomas, or the metastasis of cancer cells throughout the body. Unchecked cell growth and/or immortality are problematic biological mechanisms common to almost all types of cancer.

Another biological mechanism that is common to, and problematic in the prevention or treatment of, all solid cancer tumors is angiogenesis. Angiogenesis refers to the process by which new blood vessels are formed in the body. Without a dedicated blood supply, solid tumors have only limited growth potential—perhaps 2 mm in diameter maximum. However, angiogenesis often occurs in cancerous tissues and tumors, thus enabling solid tumors to sequester greater amounts of nutrients from the body and allowing them to proliferate rapidly, even spreading to other parts of the body. Angiogenesis is a critical means by which solid tumors grow rapidly and metastasize, hastening the process of death or disfigurement.

These two independent biological mechanisms are the common, primary modalities by which almost all cancer cells proliferate and grow. Hence, a novel approach for the treatment of cancer would be the development of pharmacological agents that have dual roles as anti-angiogenic as well as pro-apoptotic agents. Such an agent will have the ability to target both components of a cancer: kill the tumor cell by induction of apoptosis and cut off the blood supply to the tumor cell so that it will not grow.

In addition to the dire need for effective treatment modalities for existing cancers, there is arguably an even greater need for effective cancer preventative means, both with respect to initial onset of cancers, as well as in the context of recurrence of cancers after operative intervention.

A recent breakthrough in the treatment of cancer is the use of 2-methoxyoestradiol (hereinafter “2-ME”). 2-ME is an endogenous non-toxic metabolic byproduct of estrogens that is present in human urine and blood. (1) A potential role for 2-ME as a chemopreventive agent has been reported in the mammary and pancreatic models. (2) 2-ME has also been shown to inhibit endothelial cell proliferation implicating its potential role in angiogenesis. (3) In addition, apoptosis has been implicated as a mechanism for 2-ME's cytostatic and anti-angiogenic effect.

### SUMMARY OF THE INVENTION

It is an object of the present invention to provide a new modality for the treatment of cancers.

It is another object of the present invention to provide a new modality for the prevention of cancers.

It is another object of the present invention to provide a new modality for the suppression of recurrence of cancers once treated.

It is an object of the present invention to provide a new modality for the treatment of prostate cancer.

It is another object of the present invention to provide a new modality for the prevention of prostate cancer.

It is another object of the present invention to provide a new modality for the suppression of recurrence of prostate cancer once treated.

It is another object of the present invention to provide a method by which the known substance of 2-ME may be employed in a new and unobvious manner in the treatment of cancers.

It is another object of the present invention to provide a method by which the known substance of 2-ME may be employed in a new and unobvious manner in the prevention of initial onset of cancers including prostate cancer.

It is another object of the present invention to provide a method by which the known substance of 2-ME may be employed in a new and unobvious manner in preventing the recurrence of cancers, including prostate cancer, once treated.

It is another object of the present invention to provide a method by which the known substance of 2-ME, alone, or in combination with synergistic compounds, including eugenol and certain other herein disclosed compounds, may be employed in a new and unobvious manner in the treatment of cancers.

It is another object of the present invention to provide a method by which the known substance of 2-ME, alone, or in combination with synergistic compounds, including eugenol and certain other herein disclosed compounds, may be employed in a new and unobvious manner in the prevention of initial onset of cancers including prostate cancer.

It is another object of the present invention to provide a method by which the known substance of 2-ME, alone, or in combination with synergistic compounds, including eugenol and certain other herein disclosed compounds, may be employed in a new and unobvious manner in preventing the recurrence of cancers, including prostate cancer, once treated.

It is another object of the present invention to provide an agent or composition, or more than one agent or composition, that is efficacious in inhibiting the proliferation and/or angiogenesis of cancer cells.

It is another object of the present invention to provide a method for creating novel molecules that are efficacious in inhibiting the proliferation and/or angiogenesis of cancer cells.

It is another object of the present invention to provide a composition the primary active ingredient of which are an analogue or analogues of 2-methoxyestradiol which are efficacious in inhibiting the proliferation and/or angiogenesis of cancer cells.

It is another object of the present invention to provide a method for inhibiting the proliferation and/or angiogenesis of cancer cells through use of a composition the primary active ingredient of which is 2-methoxyestradiol or an analogue thereof, as described herein.

In satisfaction of these and related objects, disclosed and claimed herein is the use of 2-ME or 2ME analogues, alone, or in combination with synergistic compounds, including eugenol and certain other herein disclosed compounds, in the treatment, prevention of initial onset, and prevention of post-operative recurrence of cancers, including, but not limited to, nervous system, skin, lung, colon, liver, breast and prostate cancers.

Findings by the present inventors pertaining to the mechanisms of action of 2-ME, its derivatives, and the synergistic compounds herein described indicate that these compounds serve as cancer preventative agents, as well as curative agents. The present inventors previous work, filed with the original patent application and another continuation-in-part application, shows that 2-ME is of great significance in the treatment of prostate, brain, skin, and nervous system cancers through the induction of apoptosis. This body of work indicates that 2-ME is an anti-tumorigenic agent with a significant therapeutic advantage since it can preferentially inhibit actively proliferating cells (characteristic of tumor cells) without affecting the growth of normal cycling cells. Additionally, 2-ME appears to also inhibit the formation of new blood vessels. To the best of our knowledge, this is the first compound that targets two components of cancer: the tumor cells and their blood supply. The present inventors have demonstrated that 2-ME is a chemical compound with a significant role as an antitumorigenic agent with broad efficacy in a variety of cancerous cell populations.

Building on these findings, further experiments have helped to elucidate the structural bases for 2-ME's molecular efficacy. A number of experiments have been conducted using 2-ME and 16-epiestriol (hereinafter “16-ES”), an analogue of 2-ME that lacks the methoxy group at the second position. In a multitude of experiments, using prostate cancer cell lines (both androgen-dependent (LNCaP), and androgen-independent (DU145) cells), and a brain and/or nervous system cancer cell line (DAOY), the present inventors have studied the effects of 2-ME and 16-ES on cell proliferation and the induction of apoptosis, in a number of ways. The sum of all the data clearly indicates that 2-ME is a compound that significantly inhibits cancerous cell growth and has pro-apoptotic effects, while 16-ES does not. In total, these data show that the efficacy of 2-ME is associated with the methoxy moiety at the second position of 17β-estradiol (E₂). Further, it also indicates efficacy of a series of compounds with various moieties at the second position in the treatment of cancer. Additionally, the specific anti-proliferative, pro-apoptotic, anti-angiogenesis, and other efficacy of 2-ME against cancer cells indicates that other structural modifications of the molecule will reasonably be expected to increase the efficacy of the agent. Thus, the present inventors now propose a method of synthesizing a number of analogues of 2-ME that will exceed the efficacy of 2-ME in the prevention of cancer.

Further still, the investigations of the present inventors indicate that 2-ME (and predictably its analogs) work synergistically with other compounds, notably eugenol, to achieve even greater results in the same manner and modality as 2-ME alone in attacking cancer cells (treatment of existing cancers), in preventing initial cancer formation, and in preventing the recurrence of cancer.

### DETAILED DESCRIPTION OF THE PREFERRED EMBODIMENT

The present inventors have tested the use of 2-ME in prostate cancer prevention in an in vitro system, and have determined that there exists proven efficacy in this regard. This testing has involved the use of androgen-dependent (LNCaP) and androgen-independent (DU145 and PC-3) cell lines to investigate the effect of 2-ME.

The cells were treated with different concentrations of 2-ME (0.5 to 5 μM) and cell growth, cell cycle progression and expression of p53 was monitored every 24 hours to understand the stage at which 2-ME acts on cancer cells.

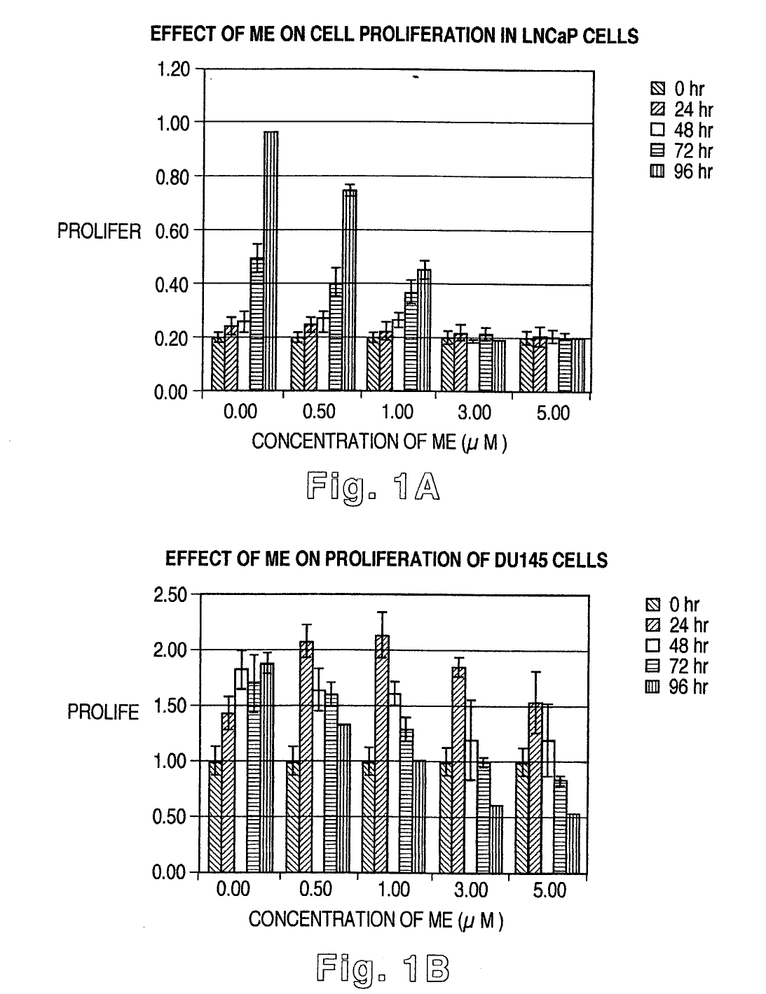

Referring to FIG. 1, actively growing LNCaP and DU145 cells were plated in 96-well plates at a density of 10⁵ cells per well. After 24 hours in a 37° C. incubator with 5% CO₂, the cells were treated with the indicated concentration of 2-ME. Control cells received only the vehicle (DMSO). Cell growth was monitored every 24 hours using the CELLTITER96 AQUEOUS ONE solution assay containing a tetrazolium compound (Promega Corporation, Madison, Wis.).

The assay is based on the principle that actively growing cells generate reducing equivalents such as NADH that is necessary for the cells to reduce the tetrazolium compound to formazan product that is detected by measuring the absorbance at 570 nm. Increase in absorbance indicates cell viability.

The data reflected in FIGS. 1A and 1B represents an average of five replicates and the experiment was conducted twice. As shown in FIGS. 1A and 1B, the control cells continued to proliferate during the time course of the experiment, the cells treated with 2-ME showed a dose-dependent inhibition of cell proliferation. The androgen-dependent LNCaP (FIG. 1A) cell line was found to be more sensitive to the effect of 2-ME than the androgen-independent DU145 cell line (FIG. 1B). It is known that there are differences of AR and p53 between these two cell types.

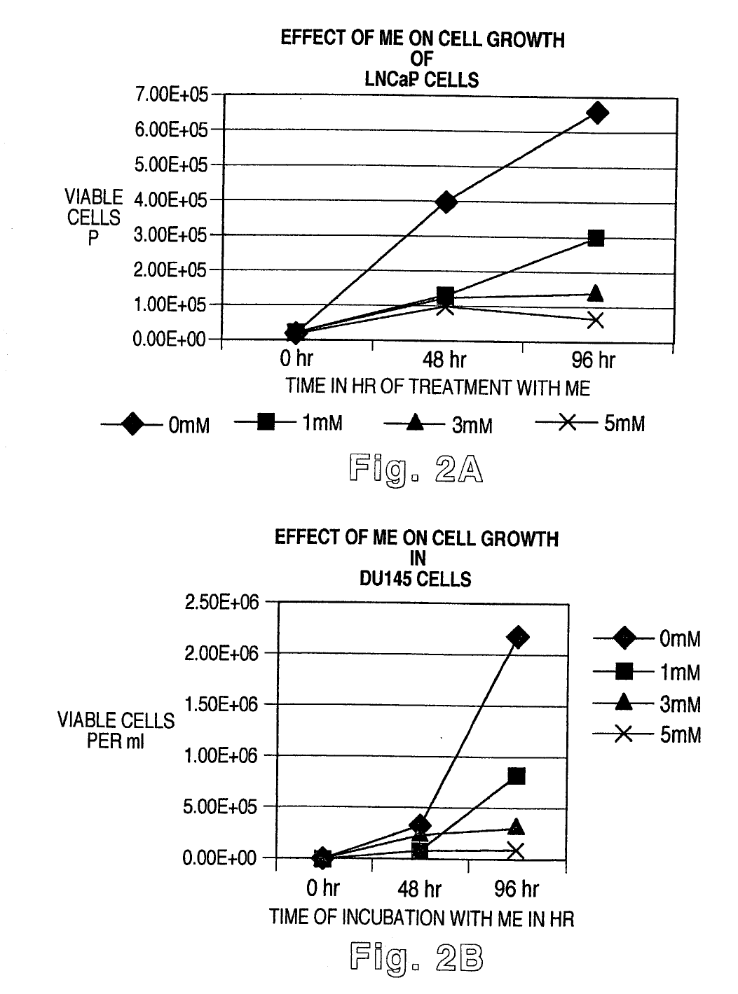

Referring to FIGS. 2A and 2B, the above experiment was confirmed by measuring cell viability using the trypan blue stain. LNCaP cells (FIG. 2A) were grown in RPMI 1640 medium; and DU145 (FIG. 2B) cells were grown in MEM Earles medium containing 10% Fetal bovine serum and penicillin and streptomycin (Life Technologies, Inc. Baltimore). Cells were plated at a density of 10⁵ cells per 24 mm dish and after 24 hours, cells were treated with the indicated concentrations of 2-ME. Cell growth was monitored by harvesting the cells at the indicated time intervals following treatment with 2-ME using trypan blue exclusion method. The cell pellet was resuspended in 0.5 ml of 0.4% trypan blue solution and after 15 minutes of incubation at RT, viable cells (unstained) were counted using a hemacytometer. The data reflected in FIG. 2 represents an average of three replicate dishes.

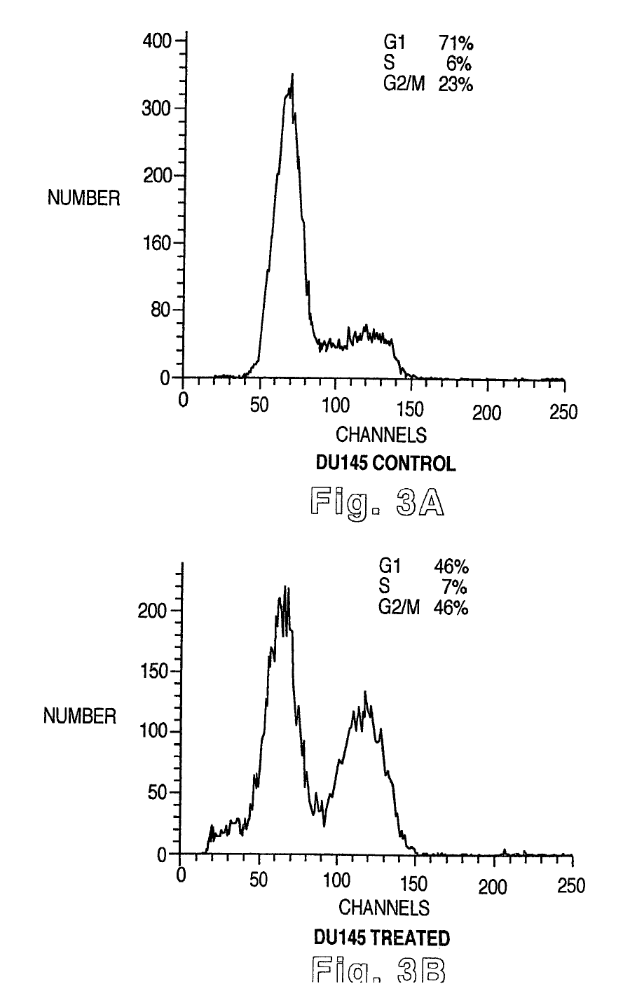

Referring to FIGS. 3A and 3B, since 2-ME inhibited the growth of LNCaP and DU145 cells, the present inventors set out to examine whether 2-ME treatment altered the distribution of cells during cell cycle progression. Logarithmically growing DU145 cells were plated in 60 mm dishes as described above at a density of 10⁵ cells per dish. After 24 hours of growth at 37° C. in an incubator with 5% CO₂, half the dishes were treated with 3 μM of 2-ME (FIG. 3B) and the other half was left untreated (FIG. 3A). Cells were observed every 24 hours for morphological changes following treatment with 2-ME. After 16 hours of incubation cells were harvested by trypsinization and the cell pellet was resuspended in 1 ml of Krishan stain and subjected to flow cytometric analysis at the Flow Cytometry facility of the University of Colorado Comprehensive Cancer Center, Denver, Colo. Flow cytometric analysis of the DU145 cells treated with 2-ME showed an increase in the G2/M population from 23% to 46% following treatment and a decrease in G1 population from 71% to 46% with no significant change in the population of cells in S phase. This data suggests that 2-ME inhibits growth of DU145 cells by arresting the cells predominantly in the G2/M phase. This could be due to alteration of expression and/or activities of cell cycle regulatory proteins in the G2/M phase.

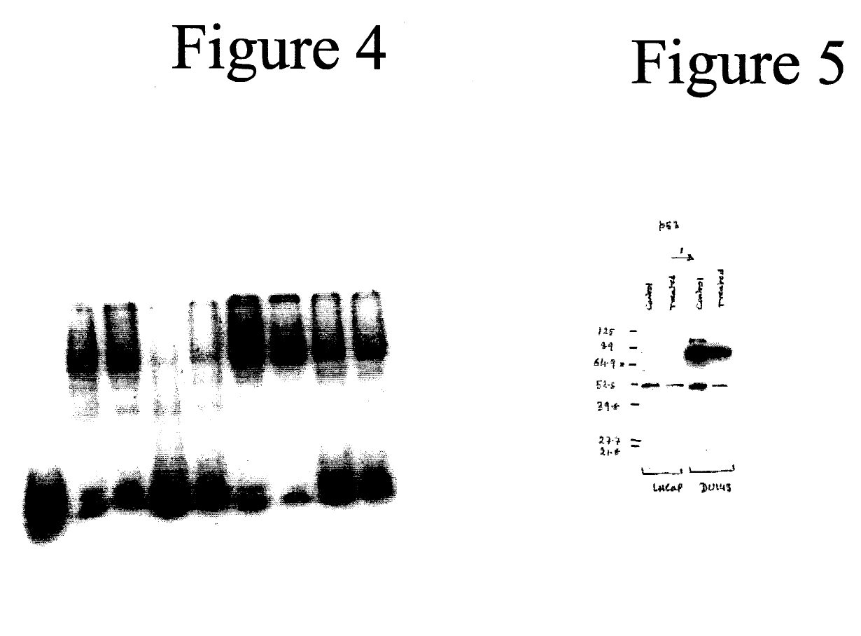

FIG. 4 depicts an electrophoretic mobility shift assay (EMSA) of whole cell extracts (prepared from LNCaP and DU145 cells that were untreated (C) or treated (T) with 3 μM 2-ME for 48 h) using p53 consensus oligonucleotide as radiolabled probe. The indicated amounts (μg) of the extract was incubated with approximately 0.2 ng of labeled probe and the DNA-protein complexes were resolved by 4% non-denaturing gel electrophoresis. Unbound (F) and bound complexes (B) are indicated. Lane 1 is free probe; lane 2, 4, 6 and 8 are with 2.5 μg protein; lanes 3, 5, 7 and 9 are with 5 μg protein. Lanes 2, 3, 6 and 7 are untreated; lanes 4, 5, 8 and 9 are treated.

FIG. 5 depicts a Western blot analysis of whole cell extracts from LNCaP and DU145 cells following treatment with 2-ME for 48 hours using a p53 antibody (FL-393, Santa Cruz). 25 μg of extract was fractionated on 10% denaturing gel and transferred to nitrocellulose membrane. After blocking the membrane, it was incubated for 2 hours with the p53 antibody. This was followed by incubation with horseradish peroxidase-conjugated anti-rabbit IgG antibody (Sigma) in the blocking solution. Bound antibody was detected by Supersignal West Pico Chemiluminescent Substrate, following the manufacturer's directions (Pierce, Rockford, Ill.).

Referring to FIGS. 4 and 5, it is well established that the tumor suppressor function of p53 is mediated by accumulation of wild type p53 in response to extracellular signals with sequential induction of either cell cycle arrest or apoptosis. Mutation of p53 which is very frequent in human cancers (50%) is the result of disruption of these signaling pathways. Deregulation of such signaling pathways ultimately provides a selective growth advantage to tumor cells. Accordingly, the present inventors tested whether 2-ME induced growth inhibition was mediated by alterations in the DNA-binding activity of p53, its levels and/or its post-translational modifications.

As shown in FIG. 4 (Electrophoretic mobility shift assay (EMSA) of extracts prepared from LNCaP and DU145 cells following treatment with 2-ME using p53 oligonucleotide probe) and FIG. 5 (Western blot analysis of extracts following 2-ME treatment with p53 antibody), the proteins from LNCaP and DU145 cells bound to the p53 consensus sequence (FIG. 4, lanes 2 and 3). Interestingly, the proteins lost their binding activity following 2-ME treatment in LNCaP extracts (FIG. 4, lanes 4 and 5) which correlates with decrease in its expression (FIG. 5, lane 2). However, the loss of DNA-binding activity of DU145 extract was not as dramatic as that of LNCaP extracts. One would expect to see induced expression of p53 following 2-ME treatment, if it is involved in mediating apoptosis.

The use of 2-ME as a chemopreventive agent and/or chemotherapeutic agent offers the following important advantages: 1) it specifically targets (inhibits the growth) actively proliferating cells (characteristic feature of cancer cells) sparing the normal or slow growing cells thus increasing its therapeutic index; and 2) the fact that 2-ME inhibits angiogenesis suggests that it can be used in the treatment of any type of cancer since all types of cancers requires the growth of blood vessels (angiogenesis). Therefore limiting blood supply would limit the spread of cancerous cells to other tissues or organs (metastasis). Unlike androgen ablation therapy where recurrence of tumors occur due to development of androgen-independence, the use of 2-ME may be advantageous since 2-ME inhibits the growth of both androgen-dependent (LNCaP) and androgen-independent (DU145) cells.

Data from the present inventors laboratory shows that 2-ME inhibits the growth of brain, nervous system and prostate cancer cells but that 16-epiestriol does not. This indicates that substituting the second position of 17b-estradiol (E₂) with a methoxy group generates a molecular structure that shows significant and selective growth inhibitory activity toward prostate cancer cells while simultaneously eliminating the potentially detrimental growth stimulating activity of E₂ itself. The analogues of 2-ME to be prepared as described below are designed (1) to determine which components of the 2-ME molecule in addition to the 2-methoxy group are required for the observed chemopreventive effects and (2) to determine if growth-inhibitory 2-ME analogues can be created that are effective.

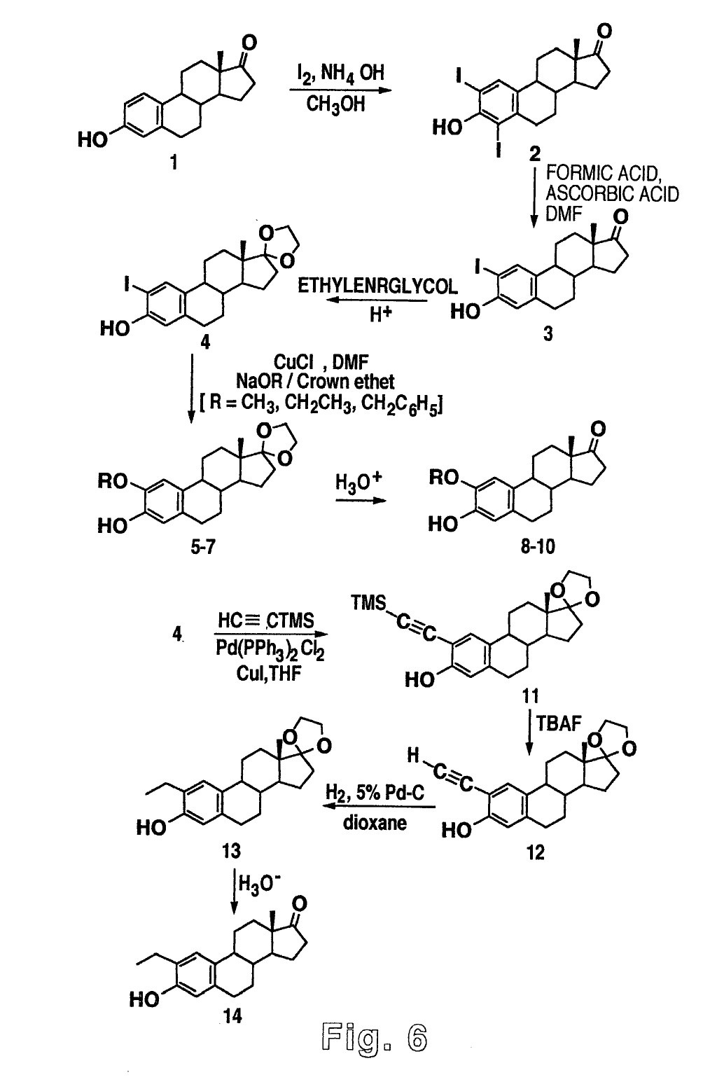

The initial compounds synthesized were 2 alkoxy substituted analogues of estrone shown in FIG. 6. These compounds will then be converted into the 2-ME analogues as shown in FIG. 8 (analogues 19-21, 23-25, and 27-29).

FIG. 6 illustrates how the A ring of the E₂ steroidal nucleus will be modified to generate 2-alkoxy substituted analogues of estrone (analogues 8-10) and a 2-ethyl substituted estrone analogue (analogue 14). The key reactions in this figure are the synthesis of compound 2,2,4-diiodoestrone, and its conversion to compound 3, the 2-iodoestrone derivative. The iodination and diodination of the estrone starting material (analogue 1) will be carried out as described by Ikegawa et al in their synthesis of catecholic equilin and equilin derivatives. (4) The proposed conversion of the ethylenedioxy protected 2-iodoestrone derivative 4 to the protected 2-methoxy, 2-ethoxy, and 2 benzyloxy derivatives 5-7 by Cu (I) catalyzed reactions of the alkoxides in dimethylformamide in the presence of a crown ether is based upon the comparable reaction of a protected 2-iodoequilin also described by Ikegawa et. al in the synthesis of catechol equilins. (4) It should be noted that if it proves necessary the estrone starting material used in FIG. 6 could be protected as the ethylenedioxy derivative by treatment with ethylene glycol prior to the iodination reaction. The Pd(Ph₃)Cl₂/CuI catalyzed coupling of the aryl iodide (analogue 4) with trimethylsilyl substituted acetylene to yield the 2-alkynyl substituted estrone derivative 11 shown in FIG. 6 has many known precedents (5). The present inventors have carried out many such coupling reactions in their laboratory and have found that molecules containing active hydrogens (NH₂ or OH groups) can be successfully coupled in such reactions if care is taken to form the reactive Cu-TMS acetylene complex before the halogenated aromatic substrate is added. It is therefore anticipated that this reaction will proceed as shown in FIG. 6. If, however, the reaction fails to be successful as shown in FIG. 6, the intermediate 4 will be coupled with trimethylsilylacetylene in 9:1 CH₃CN/H₂O catalyzed with Pd(AcO)₂/PPh₃/CuI. The present inventors have carried out a model reaction in their laboratory with an unprotected iodophenol that gave the desired coupling product with this procedure.

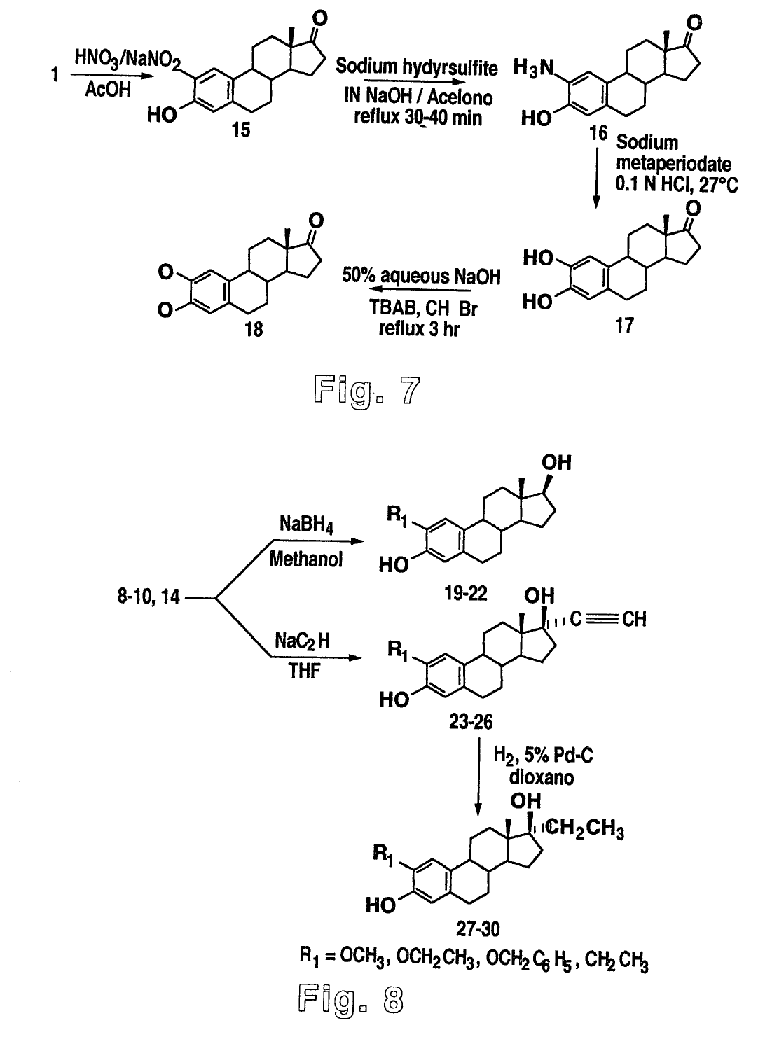

FIG. 7 outlines the reaction sequence that will be employed to prepare the 2,3-methylenedioxyestrone derivative (analogue 18). This reaction sequence is based upon the reaction sequence employed by Stubenrauch and Knuppen to prepare catechol estrogens. (6)

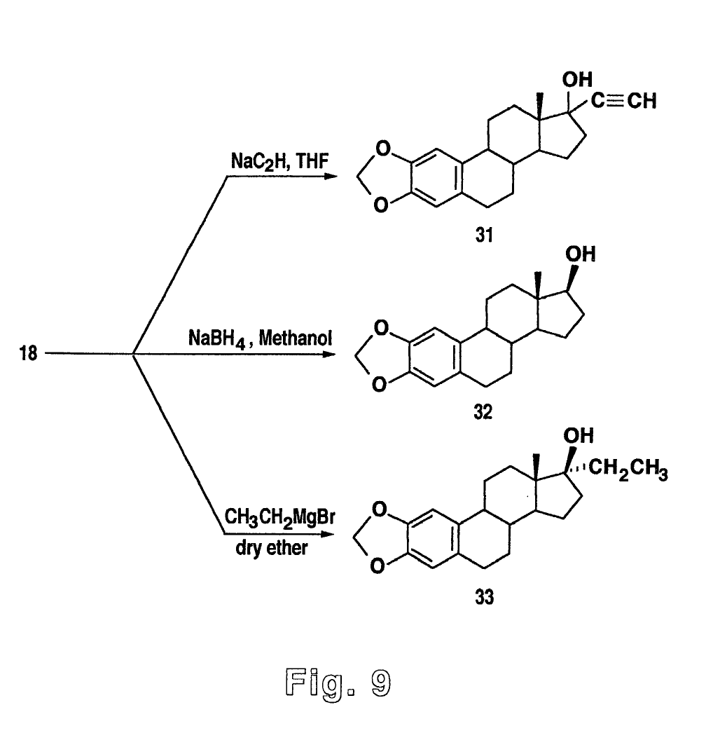

FIGS. 8 and 9 illustrate how 2-methoxyestrone and the 2-methoxyestrone analogues prepared as outlined in FIGS. 6 and 7 above will be converted into (i) 2-methoxyestrone and its analogues and (ii) 2,3-methylenedioxyestrone analogues modified at position C-17. The preparation of these structures will not only allow us to test the requirement for the 17b-hydroxyl group in the chemopreventive activity of 2-ME but will also enable us to determine if substitutions at C-17 (for example, the 17-ethynyl-2-ME derivative, 23) will decrease the rate of metabolism and deactivation of 2-ME and its analogues. As outlined in FIGS. 8 and 9 below, the present inventors propose to prepare both 2-ethyl-17b-estradiol (analogue 22) and 2,3methylenedioxy-17b-estradiol (analogue 32). In addition, since 17a-ethynylestradiol (ethynylestradiol) is both a potent estrogenic and long-lived analogue of E₂, the 17a-ethynyl derivative of 2-ME (analogue 19) will be prepared as outlined in FIG. 8. In addition, by directing synthesis to produce estrone analogues of the target structures (analogues 8-10, 14, and 18) as illustrated in FIGS. 6 and 7, it will be possible to prepare 17a-ethynyl, and 17a-ethyl derivatives of the 2-alkoxy, 2-ethyl, and 2,3-methylenedioxy analogues (analogues 23-26, 27-30, 31 and 32).

It should be noted that the proposed reactions used to modify the C-17 carbonyl of the estrone analogues shown in FIGS. 8 and 9 are standard reactions that have been successfully applied to estrone. (7)

Although not explicitly shown in FIGS. 6 and 8, the 2-ethynyl intermediate shown in FIG. 6 (analogue 12) will also be converted into 2-ethynylestrone and 2-ethynylestradiol for testing. Further, although not explicitly indicated in FIGS. 6 and 7, the 2-ethynylestrone derivative 11 shown in FIG. 6 will also be converted into 2-ethynylestrone and 2-ethynylestradiol as shown in FIG. 7 for the other intermediates. This will generate two additional 2-ME analogues for biological testing. Lastly, it is also possible to modify the acetylene coupling reaction shown in FIG. 6 to prepare 2-(1-propynyl) and 2-(1-butynyl) derivatives of 2-ME that could serve as precursors of 2-propyl and 2-butyl 2-ME analogues.

The synthesis reactions in FIGS. 6-9 outlined above will provide an efficient way of generating 2-ME (analogue 19) and fourteen 2-ME analogues (analogues 20-33) that can be utilized to determine the effects of modifying both the C-17 and the C-2 position of 2-ME. Samples of the estrone analogues themselves (analogues 8-10, 14, 18) will also be tested for their potential growth-inhibitory activity. The reaction sequences outlined in FIGS. 6-9 will therefore produce a total of 21 new 2-ME analogues to be tested as potential selective inhibitors of cancer cell growth and angiogenesis. It is anticipated that one or more of these analogues may manifest selective growth-inhibitory activities towards cancer cells while, at the same time, being less subject to metabolic conversions that will deactivate or eliminate these active analogues. It is also likely that 17a-ethynyl derivative of 2-ME may have a longer effective half-life both in vitro and in vivo.

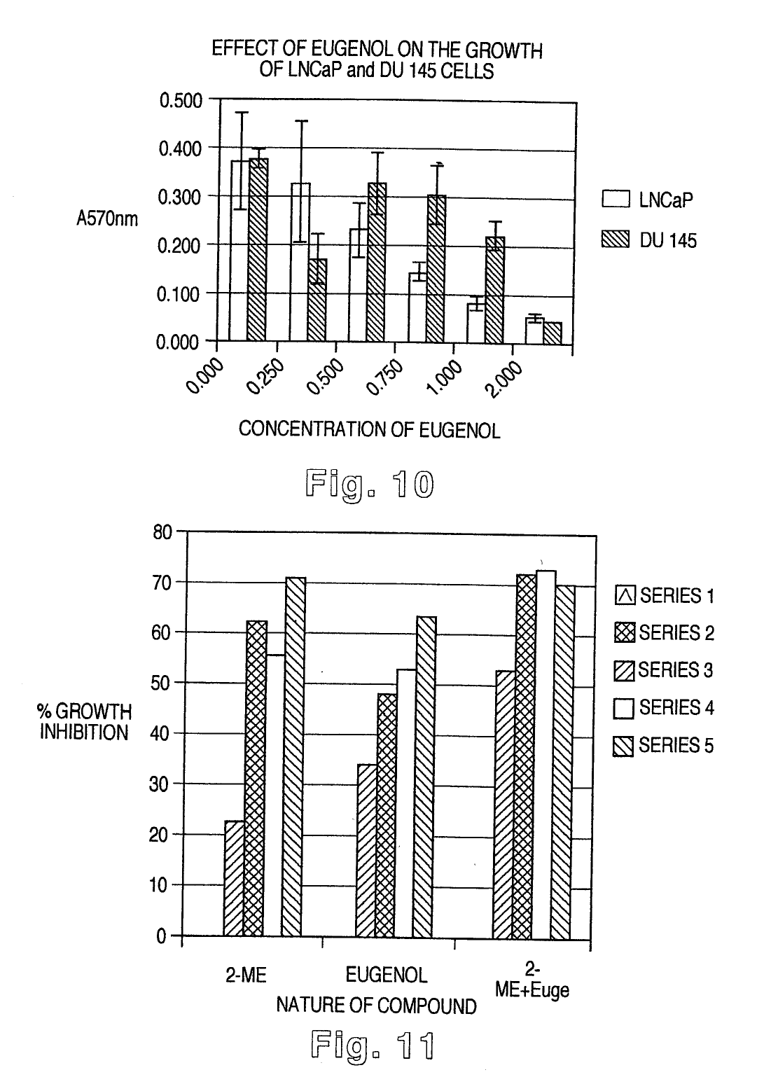

Referring to FIG. 10, eugenol also inhibits the growth of LNCaP cells significantly. A concentration of approximately 0.75 mM was necessary to see 50% inhibition of growth of LNCaP cells whereas a concentration of more than 2 mM was necessary to see similar effect in DU145 cells.

The investigational work of the present inventors establish that eugenol works in combination with 2-ME to achieve even more impressive results than either substance alone. Cells were treated with either eugenol (0.25, 0.5, 0.75 or 1 mM) or 2-ME (0.5, 1, 2 or 3 mM) or both (0.25, 0.5, 0.75 or 1 mM of eugenol along with 0.5 mM of 2-ME). Cell growth was measured following 72 hours of treatment as described above. As shown in FIG. 11, 0.5 mM of 2-ME inhibited growth of LNCaP cells by about 20% and 0.25 mM of eugenol inhibited the growth by about 30%. However, combining both the agents showed more than 50% inhibition thereby establishing a synergistic activity of eugenol and 2-ME in combating cancer cells.

To test the efficacy of 2-ME in preventing or reversing neoplastic progression, the present inventors have conducted investigations using the well-established mouse two-stage carcinogenesis model. This model was developed to study the complex multistep process of epidermal carcinogenesis since initiation and promotion are involved in the development of cancer in humans. This model is based on the finding that a sub threshold dose of a carcinogen such as 7,12-dimethylbenz(a)anthracene (DMBA) does not cause tumors unless it is followed by repetitive treatments with a tumor promoter such as TPA (12-O-tetradecanoylphorbol-13-acetate). Although treatment of mouse skin with a single large dose of DMBA can induce papillomas in about 10-20 weeks and carcinomas in about 20-60 weeks, repetitive doses are necessary to induce tumors at lower levels. No tumors are formed at a sub-threshold dose of DMBA. However, if TPA is applied to the mice initiated with a sub threshold dose of DMBA, multiple papillomas appear after a short latency period, followed by squamous cell carcinomas after a longer period. Tumors cannot be induced with repetitive treatment of TPA in the absence of initiation. Such phenotypic changes produced in the epidermis by TPA treatment are critical for neoplastic progression. One such characteristic feature of neoplastic progression is hyperplasia.

Female Sencar mice were divided into 7 groups each with 5 animals. Group I was treated with DMBA twice a week for 4 weeks; group II was treated with 2 mg of 2-ME twice a week for four weeks; group III was treated with 2-mg of 2-ME and 5 min later with DMBA twice a week for 4 weeks; group IV was treated with 2 mg of 2-ME and after 5 min DMBA was applied; TPA was applied twice a week for 4 weeks after 1 week of DMBA treatment; group V was treated with DMBA and after one week 2 mg of 2-ME and TPA were applied twice a week for four weeks; group VI was treated with DMBA and after one week treated with TPA twice a week for four weeks and group VII was treated with acetone as solvent control followed by TPA twice a week for four weeks.

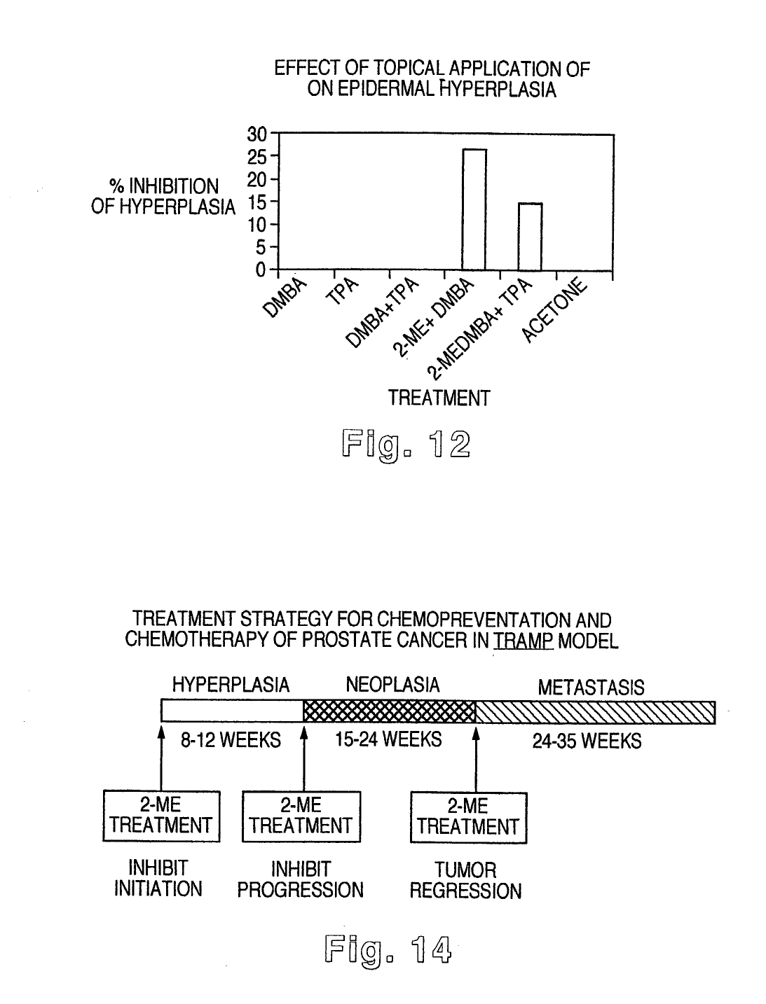

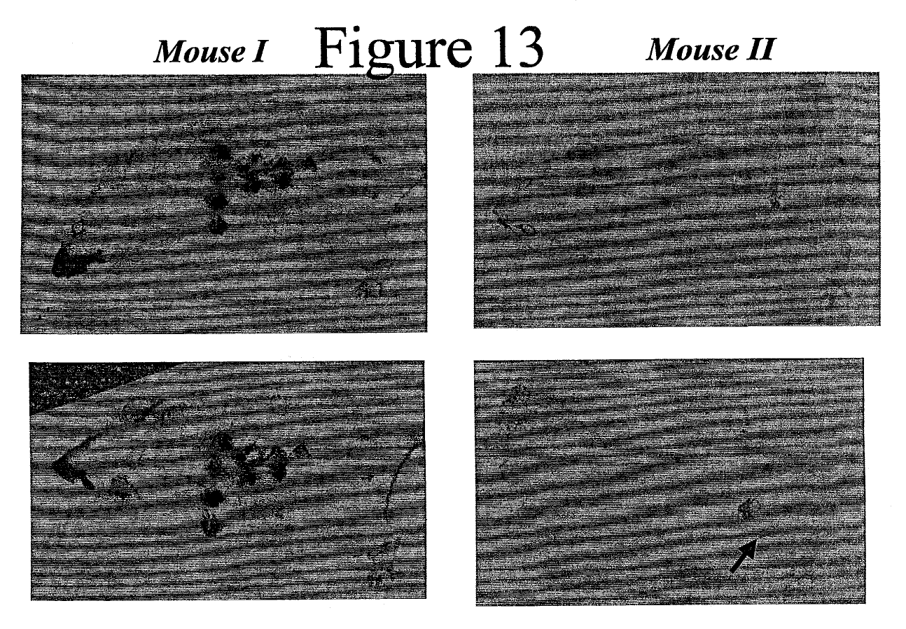

Referring to FIG. 12, after 4 weeks, animals were sacrificed and the dorsal skin was used for histology. Analysis of this data indicates that topical application of 2 mg of 2-ME 5 min before DMBA application reduced hyperplasia by 26%. In addition, topical application of 2 mg of 2-ME during tumor initiation-promotion stage inhibited hyperplasia by about 14%. These results indicate that 2-ME can be used in preventing the progression from initiated cells to the neoplastic phenotype. In addition, the present inventors have treated mice that have developed the tumors using DMBA-TPA protocol with 2 mg of 2-ME topically twice a week for four weeks to see if tumors regress. During this four-week study, we have noticed that the number of tumors reduced from 10 to 8 in one animal (one such tumor is indicated by an arrow in the mouse II—see FIG. 13) and no change in the other animal. However, we have noticed reduction in the size of the tumors. Though preliminary, these results definitely call for further investigation.

The mechanisms of action at work against the cell lines and animal models investigated thus far are reasonably expected to be equally efficacious in treating other cancers and pre-cancerous conditions, such as BPH and the cancers of brain, liver, lung, colon and skin, in preventing initial onset of cancers and pre-cancer and preventing recurrence of cancers after treatment (such as prostectomies). Since both hormone-responsive and hormone-refractory prostate cancer cells are inhibited by 2-ME and its analogs, with or without synergistic compounds such as eugenol, patients can be treated with these agents after surgery to prevent the recurrence of hormone-refractory cancer.

Additionally, the analogues of 2-ME described above are expected to provide even greater efficacy, along and in combination with synergistic, similarly structured compounds as eugenol. This expectation is well-founded on the efficacy indications established for 2-ME and the effect of the above-taught structural changes to 2-ME as indicated by the work of the present inventors.

Application to existing, in vivo tumors may be of varying means, including, but not limited to, direct injection of the herein described agents, electrophoresis, and non-electromotive transdermal migration. Practitioners skilled in the use of chemopreventative agents will adjust dosages to meet the apparent needs of any particular patient, and the disclosure contained herein shall provide an enabling disclosure for the use of 2-ME and its analogs respectively alone, and with the synergistic compound of eugenol in the prevention of cancerous tumors as well as the suppression of recurrent cancers after treatment such as surgery.

### REFERENCES

1. Gelbke, H. P., and Knuppen, R. 1976. The exertion of five different 2-hydroxyestrogen monomethyl ethers in human pregnancy urine. J Steroid Biochem. 7:457-463.

2. Zhu, B. T. and Conney, A. H. 1998. Is 2-methoxyestradiol an endogenous estrogen metabolite that inhibits mammary carcinogenesis. Cancer Res. 58:2269-2277.

3. Fotsis, T., Zhang, Y., Pepper, M. S., Adlercreutz, H., Montesano, R., Nawroth, P. P. and Schweigerer, OL. 1994. The endogenous estrogen metabolite 2-methoxyestradiol inhibits angiogenesis and suppresses tumor growth. Nature. 368:237-239.

4. Ikegawa, S., Kurosawa, T., and Tohma, M. (1988) Syntheses of C-2 caecholic equilin and equilin derivatives for use in metabolic studies. Chem. Pharm Bull. 36:2993-2999.

5. Neenan, T. X., and Whitesides, G. M. (1988) Synthesis of high carbon monomers bearing multiple ethynyl groups. J. Org. Chem., 53:2489-2496.

6. Stubenrauch, G. And Knuppen, R. (1976) Convenient large scale preparation of catechol estrogens. Steroids, 28:733-741.

7. Fieser, L. F. And Fieser, M. (1959) Estrogens in Steroids, Chapter 15, 444-502, Chapman and Hall, Ltd. London.

We claim:

1. A method for inhibiting proliferation and survival of pre-cancerous and cancerous cells comprising the steps of:
selecting a composition containing 2-methoxyestradiol; and
administering said composition to a cellular aggregation in which is identified suspected pre-cancerous or cancer cells.

2. The method of claim 1 wherein said suspected pre-cancerous or cancer cells are related to human prostate cancer.

3. The method of claim 1 wherein said suspected pre-cancerous or cancer cells are related to human nervous system cancer.

4. The method of claim 1 wherein said suspected pre-cancerous or cancer cells are related to human skin cancer.

5. A method for inhibiting proliferation and survival of pre-cancerous and cancerous cells comprising the steps of:
selecting a composition consisting substantially of one or more of 2-methoxy estradiol,2-ethoxyestradiol,2-butoxyestradiol, 17-α-ethynylestradiol
with methoxy group at position 2,17-α-ethynylestradiol with butoxy group at position 2, 17-α-ethynyl-9-α-fluoroestradiol with methoxy group at position 2; and 17-α-ethynyl-9-α-fluoroestradiol with butoxy group at position 2.
administering said composition to a cellular aggregation in which is identified suspected said pre-cancerous or cancerous cells.

6. The method of claim 5 wherein said suspected pre-cancerous or cancerous cells are prostatic cancer cells.

7. A composition for application to cellular aggregation containing pre-cancerous or cancerous cells consisting in active constituents substantially of one or more agents chosen from a groups consisting of 2-methoxyestradiol,2-ethoxyestradiol,2butoxyestradiol, 17-α-ethynylestradiol with methoxy group at position 2, 17-α-ethynylestradiol with butoxy group at position 2, 17-α-ethynyl-9-α-fluoroestradiol with methoxy group at position 2; and 17-α-ethynyl-9-α-fluoroestradiol with butoxy group at position 2.

8. The composition of claim 7 wherein said pre-cancerous or cancerous cells are related to prostate cancer.

9. The use of compositions useful in the inhibition of pre-cancerous or cancerous cell proliferation and cell survival selected from a group consisting essentially of:
the 2-ethyl-17-β-estradiol molecules identified as analogues 20-22 in FIG. 8, specifically excluding any claim to 2-methyloxyestradiol;
the 17-α-ethynyl molecules identified as analogues 23-26 in FIG. 8;
the 17-α-ethyl molecules identified as analogues 27-30 in FIG. 8;
the 2,3-methylenedioxy molecules identified as analogues 31, 32, and 33 in FIG. 9;
the 2-alkoxy substituted analogues of estrone molecules identified as analogues 8-10 in FIG. 6;
the 2-ethyl substituted molecule identified as analogue 14 in FIG. 6; and
the 2,3-methylenedioxyestrone molecule identified as analogue 18 in FIG. 7.

10. The method of claim 9 wherein said pre-cancerous or cancerous cells are related to brain cancer.

11. The method of claim 9 wherein said pre-cancerous or cancerous cells are related to prostate cancer.

12. The method of claim 9 wherein said pre-cancerous or cancerous cells are related to skin cancer.

13. The method of claim 9 wherein said pre-cancerous or cancerous cells are related to lung cancer.

14. The method of claim 9 wherein said pre-cancerous or cancerous cells are related to colon cancer.

15. A method for inhibiting pre-cancerous or cancerous cell proliferation comprising the steps of:
selecting a composition from the group consisting of the 2-ethyl-17-β-estradiol molecules identified as analogues 20-22 in FIG. 8, specifically excluding any claim to 2-methyloxyestradiol, the 17-α-ethynyl molecules identified as analogues 23-26 in FIG. 8, the 17-α-ethyl molecules identified as analogues 27-30 in FIG. 8, the 2,3-methylenedioxy molecules identified as analogues 31, 32, and 33 in FIG. 9, the 2-alkoxy substituted analogues of estrone molecules identified as analogues 8-10 in FIG. 6, the 2-ethyl substituted molecule identified as analogue 14 in FIG. 6, or the 2,3-methylenedioxyestrone molecule identified as analogue 18 in FIG. 7; and
administering said composition to cells in which is identified suspected pre-cancerous or cancer cells.

16. The method of claim 15 wherein said suspected pre-cancerous or cancerous cells are related to cancers of the nervous system.

17. The method of claim 15 wherein said suspected pre-cancerous or cancerous cells are related to prostate cancer.

18. The method of claim 15 wherein said suspected pre-cancerous or cancerous cells are related to pernicious mitosis of skin cells.

19. The method of claim 15 wherein said suspected pre-cancerous or cancerous cells are related to cancers of the colon.

20. A composition for application to pre-cancerous or cancerous cells consisting in active constituents substantially of one or more agents chosen from the 2-ethyl-17-β-estradiol molecules identified as analogues 20-22 in FIG. 8, specifically excluding any claim to 2-methyloxyestradiol, the 17-α-ethynyl molecules identified as analogues 23-26 in FIG. 8, the 17-α-ethyl molecules identified as analogues 27-30 in FIG. 8, the 2,3-methylenedioxy molecules identified as analogues 31, 32, and 33 in FIG. 9, the 2-alkoxy substituted analogues of estrone molecules identified as analogues 8-10 in FIG. 6, the 2-ethyl substituted molecule identified as analogue 14 in FIG. 6, or the 2,3-methylenedioxyestrone molecule identified as analogue 18 in FIG. 7.

21. The method of claim 20 wherein said pre-cancerous or cancerous cells are related to brain cancer.

22. The method of claim 20 wherein said pre-cancerous or cancerous cells are related to skin cancer.

23. The method of claim 20 wherein said pre-cancerous or cancerous cells are related to human prostate cancer.

24. A method for preventing the onset of cancer and for preventing the recurrence of cancer comprising the administration of a therapeutic dose to a human recipient of one or more compositions selected from the group consisting of:
2-methoxyestradiol;;
the 2-ethyl-17-β-estradiol molecules identified as analogues 20-22 in FIG. 8, specifically excluding any claim to 2-methyloxyestradiol;
the 17-α-ethynyl molecules identified as analogues 23-26 in FIG. 8;
the 17-α-ethyl molecules identified as analogues 27-30 in FIG. 8;
the 2,3-methylenedioxy molecules identified as analogues 31, 32, and 33 in FIG. 9;
the 2-alkoxy substituted analogues of estrone molecules identified as analogues 8-10 in FIG. 6;
the 2-ethyl substituted molecule identified as analogue 14 in FIG. 6; and
the 2,3-methylenedioxyestrone molecule identified as analogue 18 in FIG. 7.

25. The method of claim 24 wherein a therapuetic dose of eugenol is administered in conjunction with said one or more compositions.

26. The method of claim 24 wherein said cancer is human prostate cancer.

27. The method of claim 24 wherein said cancer is human nervous system cancer.

28. The method of claim 24 wherein said cancer is human skin cancer.

29. The method of claim 24 wherein said cancer is human colon cancer.

30. The method of claim 25 wherein said cancer is human prostate cancer.

31. The method of claim 25 wherein said cancer is human nervous system cancer.

32. The method of claim 25 wherein said cancer is human skin cancer.

33. The method of claim 25 wherein said cancer is human colon cancer.

34. A method of inducing apoptosis in cancerous tissues comprising the steps of:
administering a therapeutic dosage of a composition containing 2-methoxyestradiol to a cancerous tissues, said administration continuing at least until the initiation of cell apoptosis in said cancerous tissues.

35. A method for arresting growth of cancer tissues comprising the steps of:
administering a therapeutic dosage of a composition containing 2-methoxyestradiol to a cancerous tissue, said administration occurring at a time which, at least for some cells in said cancerous tissue, precedes cell division in the G2/M phase.
